(19) United States
(12) Patent Application Publication
(10) Pub. No.: US 2026/0027326 A1
(43) Pub. Date: Jan. 29, 2026

(54) Vascular Access System With Secondary Catheter Having Reinforced Proximal End Retention Feature

(71) Applicant: Becton, Dickinson and Company, Franklin Lakes, NJ (US)

(72) Inventor: Jonathan Karl Burkholz, Salt Lake City, UT (US)

(21) Appl. No.: 18/781,177

(22) Filed: Jul. 23, 2024

(51) Int. Cl. A61M 25/00; A61M 39/10

(52) U.S. Cl. CPC A61M 25/005; A61M 39/10

(57) ABSTRACT

An extension set for introducing an instrument into a patient's vascular system via a vascular access device including a primary catheter is provided. The extension set includes an extension tube comprising a proximal end, a distal end, and an outer tube wall that defines a tube lumen and a distal connector positioned at the distal end of the extension tube. An instrument is disposed within the tube lumen and comprises a proximal end and a distal end, the instrument movable within the tube lumen to an advanced position where the distal end of the instrument extends into the primary catheter or beyond a distal tip thereof. The proximal end of the instrument comprises a retaining feature having a first outer diameter greater than a second outer diameter of the distal end of the instrument, the retaining feature comprising a reinforcing structure that prevents deformation of the retaining feature.

### BACKGROUND OF THE INVENTION

### Field of the Invention

The present disclosure relates generally to a vascular access system with a secondary catheter and, more particularly, to a secondary catheter having improved proximal end reinforcement and delivery features that enable advancement thereof into an indwelling catheter.

### Description of Related Art

Catheters are frequently utilized to administer fluids into and out of the body. Patients in a variety of settings, including in hospitals and in home care, receive fluids, pharmaceuticals, and blood products via a vascular access device (VAD) that includes such a catheter inserted into a patient's vascular system. A common VAD includes a plastic catheter that is inserted into a patient's vein, with a length of the catheter varying from a few centimeters when the VAD is a peripheral intravenous catheter (PIVC) to many centimeters when the VAD is a central venous catheter (CVC), as examples.

Recent developments in the PIVC field have led to the emergence of technologies designed to facilitate fluid delivery and/or blood draw using an in-dwelling PIVC and accompanying instrument delivery device. The main method by which these devices work is by inserting a secondary catheter, probe, tube, or other instrument (hereafter, generally referred to as “secondary catheter”) through the catheter lumen of the PIVC, with the delivery device being attached to a catheter adapter of the PIVC that provide for insertion of the secondary catheter and allowing for advancement of the secondary catheter into the in-dwelling PIVC and into the patient's vasculature. The secondary catheter may be advanced through the in-dwelling PIVC and into the patient's vasculature by a short distance (e.g., for blood sample collection) or by a longer distance (e.g., to function as a multi-lumen midline catheter).

It is recognized that there are numerous types of delivery devices that may be employed for inserting and advancing a secondary catheter, probe, tube, or other instrument into/through the catheter lumen of the PIVC. For example, some delivery devices include an extension tube housing within which the secondary catheter is positioned. In some embodiments, the secondary catheter may be advanced distally out from the extension tube housing by way of a “flush-in-place” type method-where the secondary catheter is advanced distally as a saline flushing solution is injected into the PIVC through the introducer. In other embodiments, the secondary catheter may be advanced distally out from the extension tube housing by way of a non-contact or direct contact advancement member, with the advancement member advancing the secondary catheter as it is moved distally relative to the extension tube housing. In either of these embodiments, a proximal end of the secondary catheter may comprise an expanded or outwardly tapered region, meant to retain a portion of the secondary catheter within the introducer as it is advanced distally therein.

In each of the described delivery device configurations, it is recognized that there are drawbacks associated with the means for advancing the secondary catheter and/or the construction of the secondary catheter itself. One potential drawback for a “flush-in-place” type introducer is the risk that the secondary catheter may separate from the extension tube housing during flushing (i.e., may be flushed out from the extension tube housing, through a distal connector thereof), such as due to collapsing/deformation of the expanded region, and this may result in the secondary catheter being flushed into the vasculature of the patient, which would require surgical intervention to remove the secondary catheter. Another potential drawback for a “flush-in-place” type delivery device is the unreliability/inconsistency of the flush advancement itself, as numerous flush attempts and a significant flush volume may be required to advance the secondary catheter by a desired amount. A potential drawback for a delivery device with a non-contact advancement member is that expanded proximal end of the secondary catheter may potentially deform/collapse when pressure is applied thereto by the advancement member, which again may result in the secondary catheter potentially being able to exit the extension tube housing (through the distal connector thereof).

Therefore, it is desirable to provide a secondary catheter for use with a delivery device, where the secondary catheter is reinforced so as to prevent the secondary catheter from becoming deformed and potentially separated from the extension tube housing of the introducer. A reinforced proximal end of the secondary catheter would be sized to prevent passage thereof out through a distal connector of the extension tube housing.

### SUMMARY OF THE INVENTION

Provided is an instrument delivery device for introducing an instrument into a patient's vascular system via a catheter assembly including a primary catheter. The instrument delivery device includes an extension tube comprising a proximal end, a distal end, and an outer tube wall that defines a tube lumen. The instrument delivery device also includes an instrument disposed within the tube lumen and comprising a proximal end and a distal end, the instrument movable within the tube lumen to an advanced position where the distal end of the instrument extends into the primary catheter or beyond a distal tip thereof. The proximal end of the instrument comprises a retaining feature having a first outer diameter greater than a second outer diameter of the distal end of the instrument, the retaining feature comprising a reinforcing structure that prevents deformation of the retaining feature.

In one aspect or embodiment, the instrument comprises a secondary catheter defining a catheter lumen extending between the proximal end and the distal end thereof.

In one aspect or embodiment, the retaining feature comprises a tapered portion that flares radially outward from the second outer diameter to the first outer diameter, with the tapered portion having an increased wall thickness greater than a wall thickness of a remainder of the secondary catheter, thereby forming the reinforcing structure.

In one aspect or embodiment, the tapered portion having the increased wall thickness forms a funnel structure defining a shaped opening into the catheter lumen.

In one aspect or embodiment, the reinforcing structure comprises a stiffening component formed of a stiffening material having a stiffness greater than a stiffness of material from which the secondary catheter is formed.

In one aspect or embodiment, the stiffening component is affixed to the secondary catheter via one of adhesive bonding, welding, or swaging.

In one aspect or embodiment, the retaining feature comprises a tapered portion that flares radially outward from the second outer diameter to the first outer diameter, and wherein the stiffening component is positioned within the catheter lumen, in the tapered portion.

In one aspect or embodiment, the stiffening component defines an opening into the catheter lumen, the opening having a reduced opening area as compared to an opening arca defined by the tapered portion.

In one aspect or embodiment, the stiffening component forms one of a wedge-shaped stiffening member, a ring-shaped stiffening member, and a frustoconical-shaped stiffening member.

In one aspect or embodiment, the stiffening component is positioned about the secondary catheter.

In one aspect or embodiment, at least one of the retaining feature and the stiffening component is tipped, to provide a radius or chamfer at an end thereof.

In one aspect or embodiment, the instrument delivery device further includes a distal connector positioned at the distal end of the extension tube to connect the extension tube to the catheter assembly.

In one aspect or embodiment, the instrument delivery device further includes a proximal connector positioned at the proximal end of the extension tube, the proximal connector configured to engage a fluid delivery device that delivers fluid to the catheter assembly through the secondary catheter.

In one aspect or embodiment, the instrument is configured to move between the retracted position and the advanced position responsive to injection of a flushing fluid through the proximal connector.

In one aspect or embodiment, the instrument delivery device further includes a translation handle coupled to an outer surface of the outer tube wall, wherein the translation handle is configured to move along the outer surface of the outer tube wall between a proximal position and a distal position to translate the distal end of the instrument between a retracted position and the advanced position.

In one aspect or embodiment, the translation handle engages the retaining feature through the outer tube wall, to provide for movement of the instrument between the retracted position and the advanced position.

Also provided is a vascular access system that includes a catheter assembly including a catheter adapter comprising a distal adapter port, a proximal adapter port, and a side adapter port, a primary catheter coupled to the distal adapter port and configured to be inserted into a patient's vasculature, and a near patient connector coupled to the side adapter port via a first fluid conduit, the near patient connector comprising a distal connector port, a proximal connector port, and a side connector port. The vascular access system also includes an instrument delivery device, where an extension tube thereof is connected to the near patient connector at the proximal connector port.

In one aspect or embodiment, the vascular access system further includes an extension set integrated with the near patient connector at the side connector port, the extension set comprising a second fluid conduit for providing fluid to the catheter assembly.

In one aspect or embodiment, the extension tube is integrated with the near patient connector at the proximal connector port.

In one aspect or embodiment, the extension tube is coupled to the near patient connector at the proximal connector port via one of a luer connector and a lock comprising a blunted cannular and a pair of locking arms.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a vascular access system according to one aspect or embodiment of the present application, showing a retracted position of a secondary catheter;

FIG. 2 is a perspective view of the vascular access system of FIG. 1, showing an extended position of a secondary catheter;

FIG. 3 is a perspective view of an instrument delivery device of the vascular access system of FIG. 2, showing a retracted position of a secondary catheter;

FIG. 4 is a cross-sectional view of the instrument delivery device of FIG. 3;

FIG. 5 is a perspective view of the instrument delivery device of FIG. 3, showing an extended position of a secondary catheter;

FIG. 6 is a cross-sectional view of the instrument delivery device of FIG. 5;

FIG. 7 is a perspective view of a vascular access system according to one aspect or embodiment of the present application, showing a retracted position of a secondary catheter;

FIG. 8 is a perspective view of the vascular access system of FIG. 7, showing an extended position of a secondary catheter;

FIG. 9 is a perspective view of the vascular access system of FIG. 7, showing an instrument delivery device disconnected from a catheter assembly, and showing a retracted position of a secondary catheter;

FIG. 10 is a perspective view of the vascular access system of FIG. 7, showing an instrument delivery device disconnected from a catheter assembly, and showing an extended position of a secondary catheter;

FIG. 11 is a cross-sectional view of the instrument delivery device in the vascular access system of FIG. 7, showing a retracted position of a secondary catheter;

FIG. 12 is a cross-sectional view of the instrument delivery device in the vascular access system of FIG. 7, showing an extended position of a secondary catheter;

FIG. 13 is a perspective view of a vascular access system according to one aspect or embodiment of the present application, showing a retracted position of a secondary catheter;

FIG. 14 is a perspective view of the vascular access system of FIG. 13, showing an extended position of a secondary catheter;

FIG. 15 is a cross-sectional view of a proximal end of a secondary catheter included in an instrument delivery device, according to one aspect or embodiment of the present application;

FIG. 16 is a perspective view of the proximal end of the secondary catheter of FIG. 15;

FIG. 17 is a side view of the proximal end of the secondary catheter of FIG. 15, according to one aspect or embodiment of the present application;

FIG. 18 is an end view of the proximal end of the secondary catheter of FIG. 15, according to one aspect or embodiment of the present application;

FIG. 19 is an end view of the proximal end of the secondary catheter of FIG. 15, according to one aspect or embodiment of the present application;

FIG. 20 is a side view of the proximal end of a secondary catheter included in an instrument delivery device, according to one aspect or embodiment of the present application;

FIG. 21 is a side view of the proximal end of a secondary catheter included in an instrument delivery device, according to one aspect or embodiment of the present application;

FIG. 22 is a side view of the proximal end of a secondary catheter included in an instrument delivery device, according to one aspect or embodiment of the present application;

FIG. 23 is an end view of the proximal end of the secondary catheter of any of FIGS. 20-22, according to one aspect or embodiment of the present application;

FIG. 24 is an end view of the proximal end of the secondary catheter of any of FIGS. 20-22, according to one aspect or embodiment of the present application;

FIG. 25 is a cross-sectional view of a proximal end of a secondary catheter included in an instrument delivery device, according to one aspect or embodiment of the present application;

FIG. 26 is a cross-sectional view of a proximal end of a secondary catheter included in an instrument delivery device, according to one aspect or embodiment of the present application; and

FIG. 27 is a cross-sectional view of a proximal end of a secondary catheter of an instrument delivery device, positioned within an extension tube and engaged with a translation handle of the instrument delivery device, according to one aspect or embodiment of the present application.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

In the present disclosure, the distal end of a component or of a device means the end furthest away from the hand of the user (and closest to the body of a patient) and the proximal end means the end closest to the hand of the user, when the component or device is in the use position.

Aspects of the disclosure are directed to instrument delivery devices and associated vascular access systems that includes such delivery devices along with a catheter assembly that provides access to the vasculature of a patient. An instrument delivery device is configured to advance an instrument, such as a secondary catheter, into the catheter assembly and out past a distal tip of the primary indwelling catheter, to facilitate fluid delivery and/or blood withdrawal. A proximal end of the secondary catheter includes a retaining feature thereon that functions to retain the proximal end of the secondary catheter within the delivery device during advancement, with the retaining feature including a reinforcing structure that prevents deformation of the retaining feature, so as to ensure such retention of the secondary catheter.

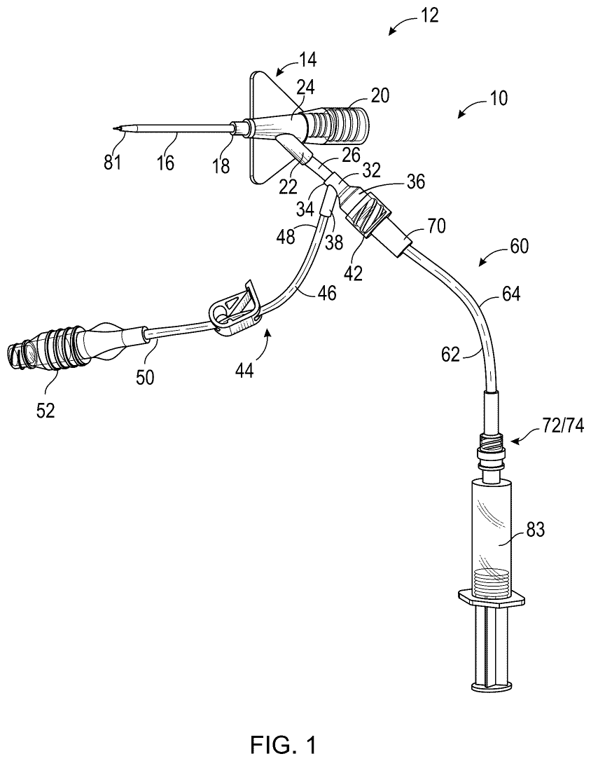

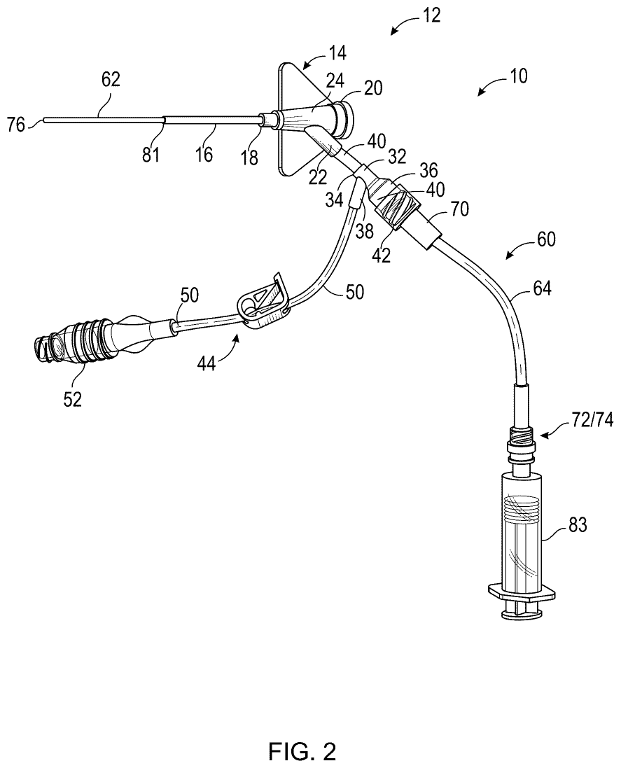

Referring to FIGS. 1 and 2, shown is a non-limiting embodiment of a vascular access system 10 for facilitating blood draw and/or administering of fluids for a patient. The vascular access system 10 may generally include a catheter assembly 12 and an instrument delivery device 60 that may be connected to the catheter assembly 12 to introduce an instrument (e.g., a secondary catheter) thereto. Connection of the instrument delivery device 60 with the catheter assembly 12 provides a vascular access system 10 with increased functionality, including extending the reach of a primary catheter (of the catheter assembly 12) into the vasculature system of a patient for short term needs (e.g., collecting a blood sample) and/or for longer term needs (e.g., converting the shorter peripheral intravenous catheter (PIVC) assembly into a multi-lumen midline catheter.

In the illustrated embodiment, catheter assembly 12 is constructed/arranged as an integrated catheter assembly that includes an integrated near-patient connector and extension set, as described in further detail below. However, it is recognized that the catheter assembly 12 may have another suitable construction, according to additional aspects of the disclosure.

As shown in FIGS. 1 and 2, the catheter assembly 12 includes a catheter adapter 14 and an associated (primary) catheter 16. The catheter adapter 14 may include a distal adapter port or end 18 and a proximal adapter port or end 20, as well as an additional or “side” adapter port 22 that may disposed between the distal end 18 and the proximal end 20. The catheter adapter 14 may include a (first) lumen 24 extending through the distal end 18 and the proximal end 20. The catheter 16 extends from the distal end 18 of catheter adapter 14 and may be configured as a PIVC that is placed into a vein of the patient, with a distal end or tip of the catheter 16 positioned appropriately within the vein to enable a blood draw from the patient. Catheter 16 may be formed of any suitable material and may be of any useful length, as known to those of skill in the art.

Referring still to FIGS. 1 and 2, in some non-limiting embodiments or aspects, the catheter assembly 12 may include a first fluid conduit 26 extending from the side port 22, with the first fluid conduit 40 integrated with the side port 22. First fluid conduit 26 may be formed of any suitable material known to those of skill in the art and may have a distal end and a proximal end. The distal end of first fluid conduit 26 is coupled to port 22, while the proximal end of first fluid conduit 26 may be coupled to a near-patient or “near port” connector 32 (hereafter “connector 32”).

As shown in FIGS. 1 and 2, according to an exemplary aspect of the disclosure, connector 32 includes a distal connector end or port 34 and a proximal connector end or port 36 thereon. The connector 32 also includes a side connector port 38 thereon that is disposed between the distal and proximal ports 34, 36. According to embodiments, the connector 32 may be configured as a t-connector (e.g., one side port arranged at a 90 degree angle relative to a longitudinal axis of connector 32) or a y-connector (e.g., one side port arranged at a 15-85 degree angle relative to a longitudinal axis of connector 32). The connector 32 includes a (second) lumen 40 therein, with the lumen 40 having any number of branches suitable for the type of connector, such as a branch extending between distal and proximal ports 34, 36 and a branch provided to the side port 38.

In accordance with some embodiments or aspects, a needleless access connector 42, which may be configured as a split-septum valve, is provided in the proximal port 36 of connector 32. The needleless access connector 42 provides a near-patient access port to the catheter assembly 12 via which external devices may be connected to catheter assembly 12, such as an instrument delivery device 50 that may introduce an instrument into the catheter assembly, as explained in further detail below.

In some non-limiting embodiments or aspects, catheter assembly 12 also includes an extension set 44 coupled to the side port 38 of connector 32. The extension set 44 may include a second fluid conduit 46 having a distal end 48 and a proximal end 50. The distal end 48 of the second fluid conduit 46 may be coupled to side port 38 of connector 32, while the proximal end 50 of the second fluid conduit 46 may include a proximal connection or port 52, i.e., a “far port connector” 52. In some embodiments, the far port 52 may comprises a luer connection to which a fluid delivery device may be connected.

The extension set 44 may be used to provide a fluid path to the catheter assembly 12 to enable fluid or medication delivery, as a non-limiting example. In some embodiments, a fluid delivery device may be coupled to the far port connector 52 to perform an intravenous infusion via a gravity flow drip, syringe push, or saline flush, as non-limiting examples.

As shown in FIGS. 1 and 2, instrument delivery device 60 is connected to catheter assembly 12 via connector 32—i.e., through needleless access connector 42 in proximal port 36. The instrument delivery device 60 is operable to introduce and advance an instrument 62 into the catheter assembly 12. According to embodiments the instrument 62 may comprise a secondary catheter, probe, guidewire, or other instrument—with the instrument 62 generally referred to hereafter as a “secondary catheter 62” for simplicity. Where the instrument 62 is a secondary catheter, the secondary catheter 62 may be constructed of a soft or flexible material to facilitate smooth placement thereof within the vasculature of the patient, with little or no damage to the vasculature. In some embodiments, the secondary catheter 62 may be constructed of silicon, polyimide, latex, polyurethane, nylon, polyethylene, or another suitable material.

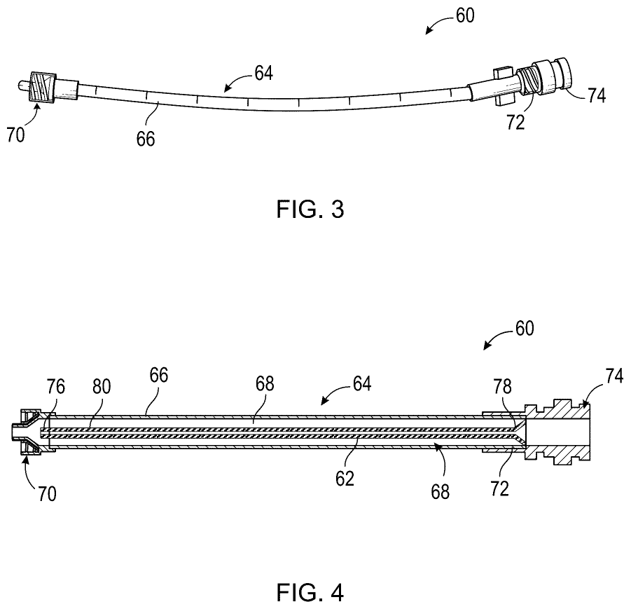

According to aspects of the disclosure, instrument delivery device 60 includes a housing 64 that, as shown in FIGS. 1 and 2 and in more detail in FIGS. 3-6, may be provided as an extension tube (hereafter, “extension tube 64”). The extension tube 64 includes a cylindrical outer wall 66 that defines an interior tube lumen 68. In some embodiments, a distal connector 70 is provided at a distal end of extension tube 64 and a proximal connector 72 is provided at a proximal end of extension tube 64. In the illustrated embodiment, distal connector 70 is a luer connector (e.g., male luer connector) configured to engage the needleless access connector 42, to connect the instrument delivery device 60 to catheter assembly 12, although distal connector 70 may be another type of suitable connector. Proximal connector 72 may also comprise a luer connector (e.g., female luer connector) and, in some embodiments, may include a needleless access connector 74 coupled thereto, to control access to the interior tube lumen 68 of instrument delivery device 60.

As shown in FIGS. 3-6, the secondary catheter 62 includes a distal end 76 and a proximal end 78, with a catheter lumen 80 extending through a length of the secondary catheter 62 between the distal end 76 and the proximal end 78. As explained in detail further below, the proximal end 78 may comprise an expanded portion (or retaining feature) thereon that functions to prevent the secondary catheter 62 from becoming separated from the instrument delivery device 60 (i.e., exiting out through distal connector 70) and/or from progressing into/through the catheter assembly 12.

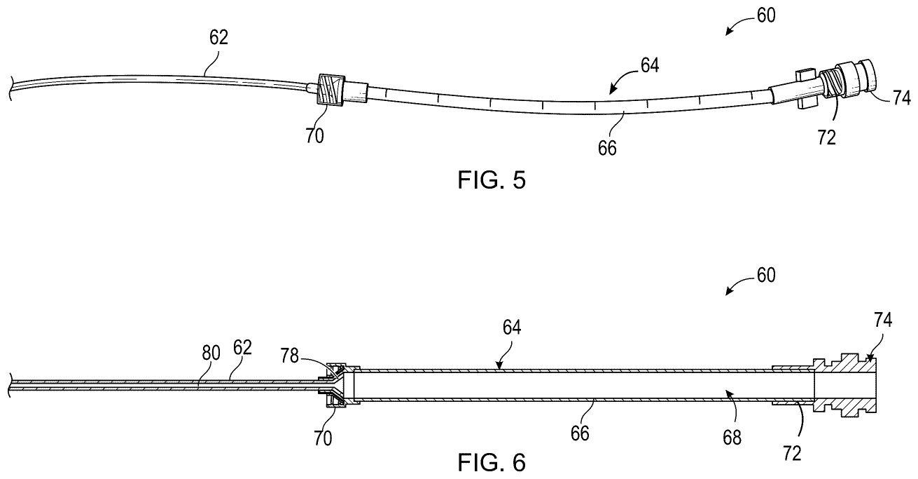

The secondary catheter 62 is housed within interior tube lumen 66 of extension tube 64 and is unattached from the extension tube 64. The secondary catheter 62 has a retracted position (FIGS. 3 and 4) where the distal end 76 of the secondary catheter 62 is positioned within the extension tube 64 and/or distal connector 70, and an extended position (FIGS. 5 and 6) where the distal end 76 of the secondary catheter 62 extends out from the extension tube 64, through the catheter assembly 12, and out beyond a distal tip 81 of the primary catheter 16. As previously described, the secondary catheter 62 is configured to extend the reach of the vascular access system 10 (and of primary catheter 16) into a patient's vascular system for short term needs, such as collecting a blood sample, as well as longer-term needs, such as providing multi-lumen mid-line catheter capability. The vascular access system 10 is configured to utilize the secondary catheter 62 for blood draw and/or administering medication, while also providing the option of simultaneously infusing fluids through the extension set 44 and delivery device 60.

In the illustrated embodiment, instrument delivery device 60 is configured to move the secondary catheter 62 from its retracted position to its extended position via injection of a flushing fluid into instrument delivery device 60 through proximal connector 72. That is, a pre-filled syringe 83 may be attached to instrument delivery device 60 via proximal connector 72, and secondary catheter 62 may be moved from its retracted position to its extended position in response to pressure resulting from depression of a plunger of the pre-filled syringe. In some embodiments, the flushing performed to advance secondary catheter 62 may be referred to as a “flush-in-place” technique because the primary catheter 16 may be dwelling within the vasculature of the patient when the flushing occurs. In some embodiments, when the secondary catheter 62 moves from the retracted position to the extended position to advance the secondary catheter 62, the distal end 76 of the secondary catheter 62 may move distally through one or more of the following in a non-tortuous fluid path: the distal connector 70, the near patient connector 32, the first fluid conduit 26, the side adapter port 22, the distal adapter port 18, and the primary catheter 16.

The vascular access system 10 of FIGS. 1-6 is configured to function similarly to a standard integrated peripheral intravenous catheter with the ability to infuse through the far port connector 70 of extension set 44 and the proximal connector 72 of instrument delivery device 60. When flushing through the proximal connector 72 of instrument delivery device 60, fluid flows through the extension tube 64 and through the secondary catheter 62 to ensure the respective lumens remain flushed (i.e., tube lumen 68 and catheter lumen 80). When the secondary catheter 62 is in the extended position, the secondary catheter 62 can be utilized for delivering caustic medication. Delivering medications further downstream in a patient's vein away from the distal tip 81 of the primary catheter 16 allows for quicker hemodilution and reduces the risk of chemical phlebitis at the location of the primary catheter 16.

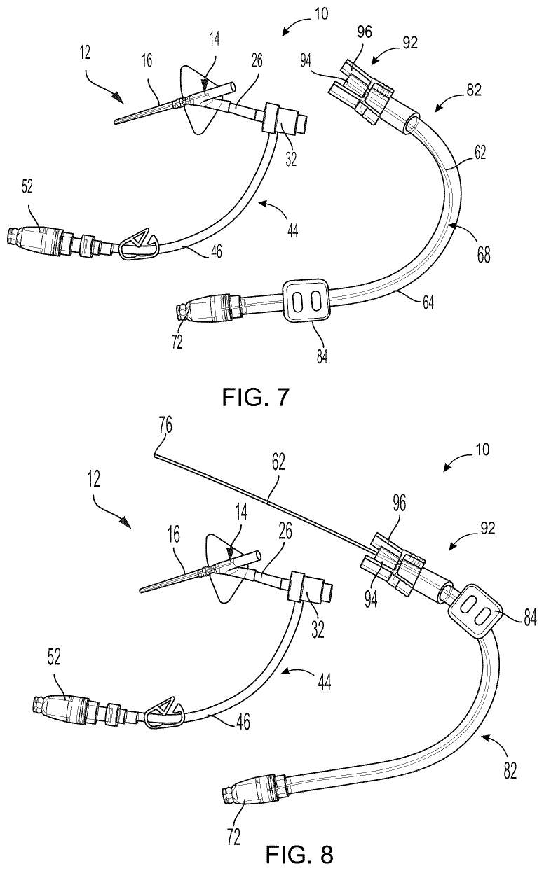

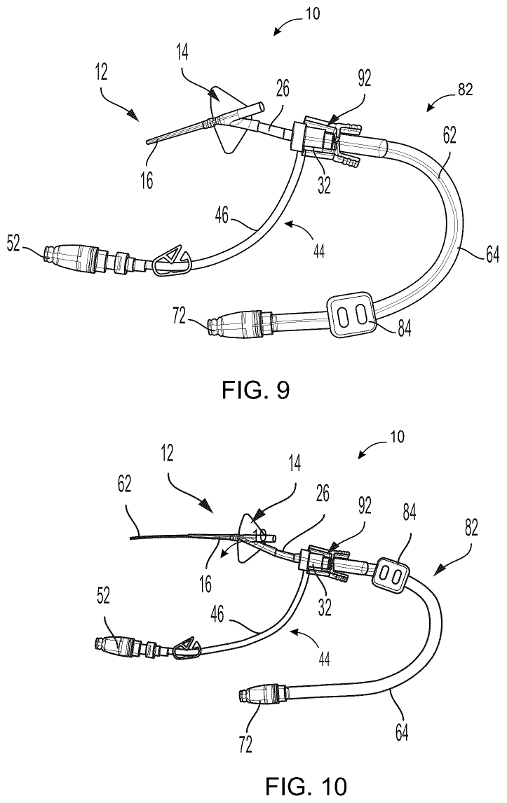

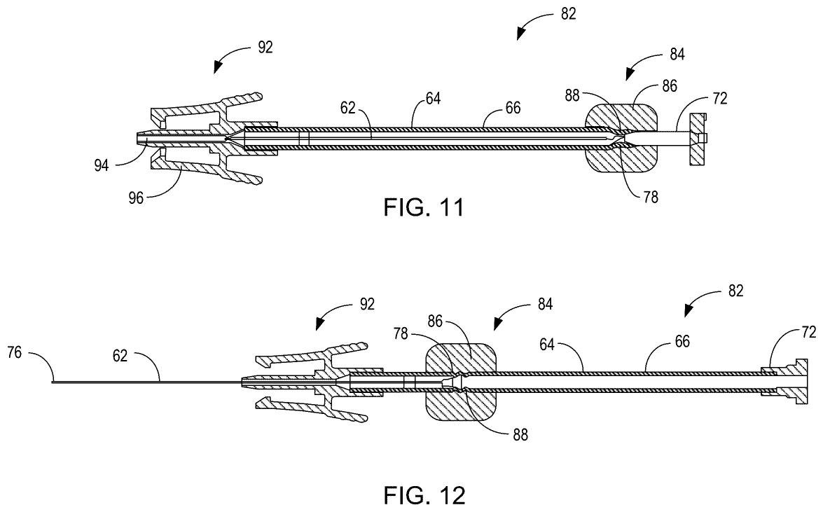

Referring now to FIGS. 7-12, an instrument delivery device 82 is shown according to another embodiment of the disclosure. The delivery device 82 of FIGS. 7-12 moves the secondary catheter 62 between the retracted position (FIGS. 7, 9, and 11) and the extended position (FIGS. 8, 10, and 12) via use of a non-contact translation handle 84 provided on the instrument delivery device 82, rather than via a fluid flushing technique as previously described with the instrument delivery device 60. The translation handle 84 is configured to move along an outer surface of the extension tube 64, with the translation handle 84 entirely positioned outside of the extension tube 64. As shown in FIGS. 11 and 12, the translation handle 84 may include a body 86 having gripping surfaces 88 that engage the outer surface of the extension tube 64. In one aspect or embodiment, gripping surfaces 88 may include bumps, such as cylindrical or spherical bumps, that engage the outer surface of the extension tube 64. The gripping surfaces 88 may engage the proximal end 78 of the secondary catheter 62 (i.e., a retaining feature at the proximal end 78, as described in detail further below) through the extension tube 64. Movement of the translation handle 84 distally or proximally causes the gripping surfaces 88 to engage the proximal end 78 of the secondary catheter 62, with the extension tube 64 depressed and positioned between the gripping surfaces 88 and the proximal end 78 of the secondary catheter 62, thereby moving the secondary catheter 62 along with the translation handle 84.

As can be seen in FIGS. 7-12, the instrument delivery device 82 may include a distal connector 92 configured as a lock that engages connector of catheter assembly 12. The lock type distal connector 92 may thus comprise a blunted cannula 94 that engages needleless access connector 42, along with a pair of locking arms 96 that aid in securing the instrument delivery device 82 to the near patient connector 32. In other embodiments, the distal connector 92 of instrument delivery device 82 may comprise a luer connector such as shown and described for the instrument delivery device 60 of FIGS. 1-6.

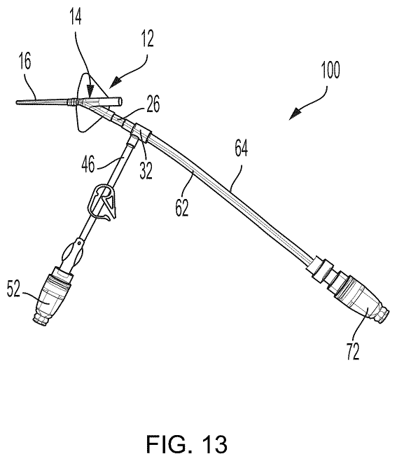

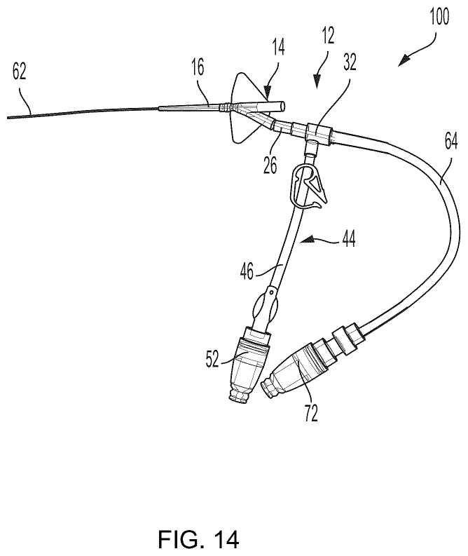

It is recognized that vascular access systems having various other configurations may be provided, according to additional aspects of the disclosure. For example, a vascular access system 10 such as shown in FIGS. 13 and 14 may be configured such that an instrument delivery device 100 thereof is integrated with the catheter assembly 12. That is, a distal end of extension tube 64 may be permanently joined (i.e., integrated) with the near patient connector 32 at proximal connector port, such as through bonding, adhesive, or another suitable arrangement. The instrument delivery device 100 in vascular access system 10 may be configured to advance the secondary catheter 62 via any of a number of suitable advancement means, such as via a flush-in-place method as previously described in the system embodiment of FIGS. 1-6 or via a non-contact translation handle 84 as previously described in the system embodiment of FIGS. 7-12.

According to aspects of the disclosure, for any of the embodiments shown in FIGS. 1-14, the proximal end 78 of secondary catheter 62 is configured to prevent the secondary catheter 62 from becoming separated from the extension tube. For the instrument delivery device 60, 100 of FIGS. 1-6 and/or FIGS. 13 and 14, this means that proximal end 78 of secondary catheter 62 is prevented from being flushed out through the extension tube 64 (and distal connector 70 for instrument delivery device 60) during injection of a flushing fluid that advances the secondary catheter 62 distally into/through catheter assembly 12. For the instrument delivery device 82 of FIGS. 7-12, this means that proximal end 78 of secondary catheter 62 is prevented from exiting out through the extension tube 64 and distal connector 92 after being distally advanced via actuation of the translation handle 84. To prevent the secondary catheter 62 from becoming separated from the extension tube and exiting out therefrom, the proximal end 78 of the secondary catheter 62 includes a retaining feature 110 therein having a (first) outer diameter that is greater than a (second) outer diameter of the distal end of the secondary catheter 62-a radially expanded portion. The (first) outer diameter of the retaining feature 110 is also greater than a (third) diameter of an opening in the distal connector 70, 92 of the instrument delivery device 60, 82. In some embodiments, the radially expanded retaining feature 110 may comprise an outwardly tapered portion 112 that increases the size/diameter of the proximal end 78 of the secondary catheter 62, while in other embodiments the radially expanded retaining feature 110 may have a stepped configuration.

According to aspects of the disclosure, in order to prevent a collapsing or deformation of the retaining feature 110 that might occur during flush-in-place or mechanical advancement of the secondary catheter 62 (and thereby allow for separation of the secondary catheter 62 from the extension tube 64), the retaining feature 110 comprises a reinforcing structure 114 that prevents collapsing/deformation of the retaining feature 110. The reinforcing structure 114 provided in/on the retaining feature 110 increases a structural rigidity and robustness of the retaining feature 110, so that the retaining feature 110 functions as intended to retain a proximal end 78 of secondary catheter 62 within the instrument delivery device 60, 82, 100 and prevent the secondary catheter 62 from passing into/through the catheter assembly 12.

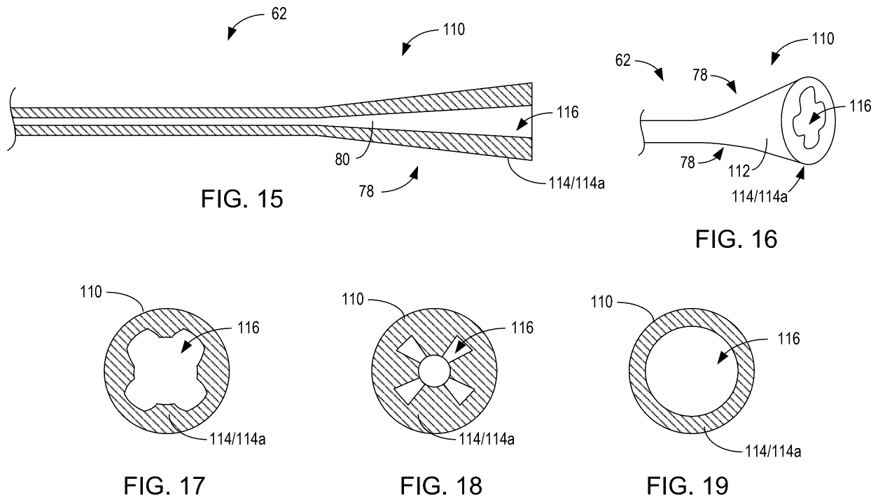

According to some embodiments, and as shown in FIGS. 15 and 16, the reinforcing structure 114 in/on retaining feature 110 is provided by forming the retaining feature 110 itself to have an increased rigidity (i.e., the reinforcing structure 114 is formed monolithically with a remainder of secondary catheter 62). For example, in embodiments where the retaining feature 110 is provided as an outwardly tapered portion 112 of secondary catheter 62, reinforcing structure 114 is provided in the form of tapered portion 112 having an increased wall thickness that is greater than a wall thickness of a remainder of the secondary catheter 62. The tapered portion 112 having the increased wall thickness may thus form a reinforced funnel structure 114a that is resistant to collapsing or other deformation that might occur during a flush-in-place or mechanical advancement of the secondary catheter 62. In some embodiments, the funnel structure 114a may be configured to present a shaped opening 116 into the catheter lumen 80 of secondary catheter 62, with exemplary shaped opening configurations illustrated in FIGS. 17 and 18, according to various embodiments. In other embodiments, the funnel structure 114a may be configured to present a circular opening 116 into the catheter lumen 80 of secondary catheter 62, as shown in FIG. 19.

According to other embodiments, the reinforcing structure 114 provided in/on retaining feature 110 is provided as a separate material/component from the secondary catheter 62. In such embodiments, the reinforcing structure 114 comprises a stiffening component 114b that is affixed/joined to or integrated with the secondary catheter 62. The stiffening component 114b may be formed from a stiffening material that may comprise a rigid polymeric material, metal, or composite having an increased hardness and rigidity as compared to a material from which the secondary catheter 62 is formed (e.g., silicon, polyimide, latex, polyurethane, nylon, polyethylene, etc., so that the catheter is flexible/bendable). Depending on the configuration of the stiffening component 114b, the stiffening component 114b may be formed, bonded, “tipped”, glued, ultrasonically welded, swaged, or otherwise joined to the retaining feature 110 on secondary catheter 62.

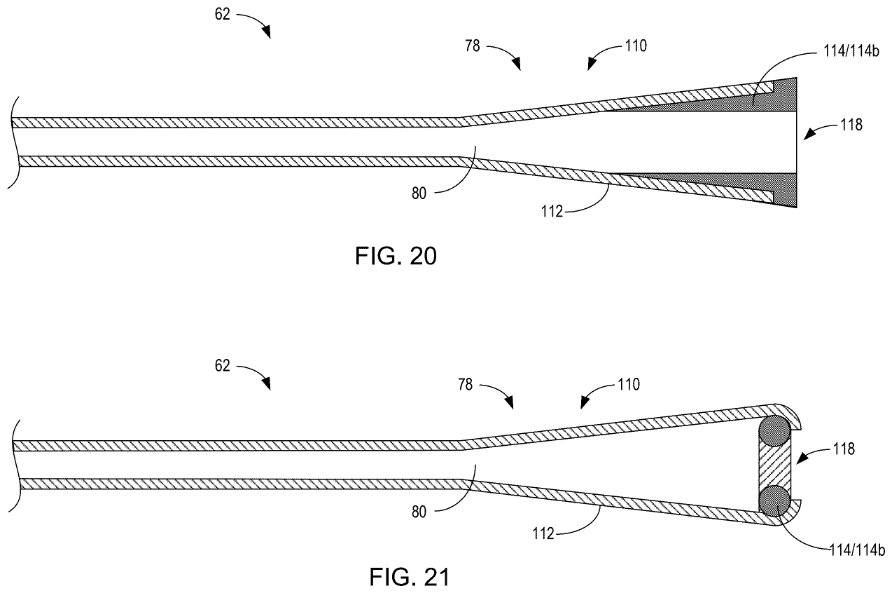

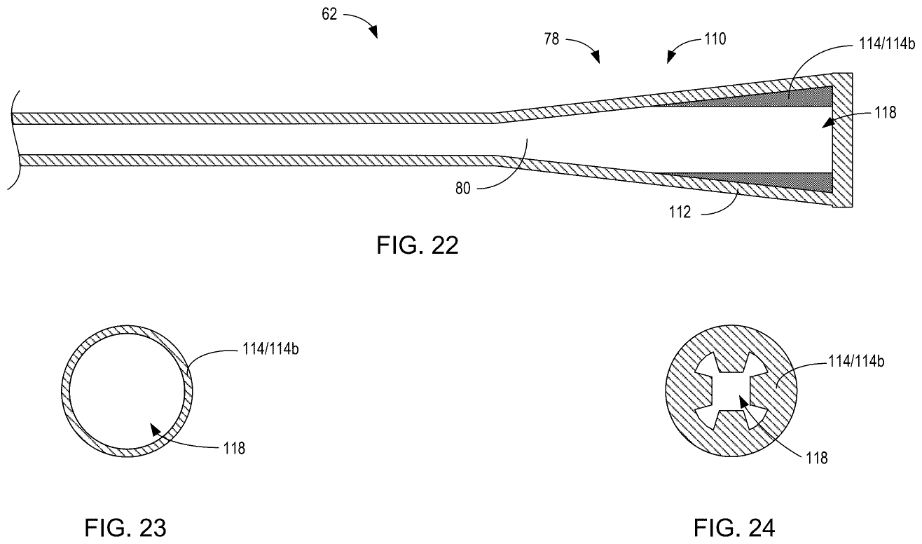

In some embodiments, where the retaining feature 110 is provided as an outwardly tapered portion 112 of secondary catheter 62, the stiffening component 114b may be provided within an inner volume defined by the tapered portion 112, such that the stiffening component 114b is seated within the tapered portion 112. As shown in FIGS. 20-22, the stiffening component 114b may be formed as a wedge-shaped stiffening member (FIG. 20), a ring-shaped stiffening member (FIG. 21), or a frustoconical-shaped stiffening member (FIG. 22), as non-limiting examples. In such embodiments, the stiffening component 114b may extend out to or past a proximal end of the tapered portion 112 (e.g., FIG. 20), or the tapered portion 112 may extend out proximally past the stiffening component 114b forming a “tubing tip” (e.g., FIG. 22).

In one aspect or embodiment, with the stiffening component 114b positioned within an inner volume defined by the tapered portion 112, the stiffening component 114b defines an opening 118 into the catheter lumen 80 having a reduced opening arca as compared to an opening area defined by the tapered portion 112. In some embodiments, the opening 118 may comprise a circular opening, such as shown in FIG. 23. In other embodiments, the opening may comprise a shaped opening, such as shown in FIG. 24.

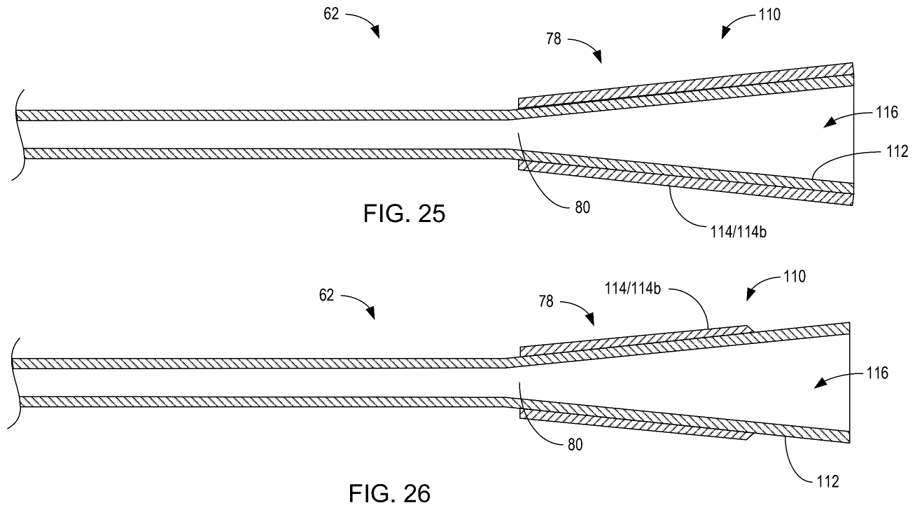

In some embodiments, the stiffening component 114b may be positioned about (i.e., around an outer surface of) at least a portion of the retaining feature 110, as shown in FIGS. 25 and 26. For example, the stiffening component 114b may be positioned on/around the outer surface of the tapered portion 112-with the stiffening component 114b thus having a frustoconical shape. The stiffening component 114b may cover an entirety of the tapered portion 112 (FIG. 25) or only part of the tapered portion 112 (FIG. 26).

In some embodiments, and depending on the structure, arrangement and/or method of assembly of the retaining feature 110 and the stiffening component 114b, at least one of the retaining feature 110 and the stiffening component 114b is tipped, to provide a radius or chamfer at an end thereof. As shown in FIG. 25 as one example, stiffening component 114b is positioned about an outer surface of retaining feature 110 and is tipped at the proximal edge thereof.

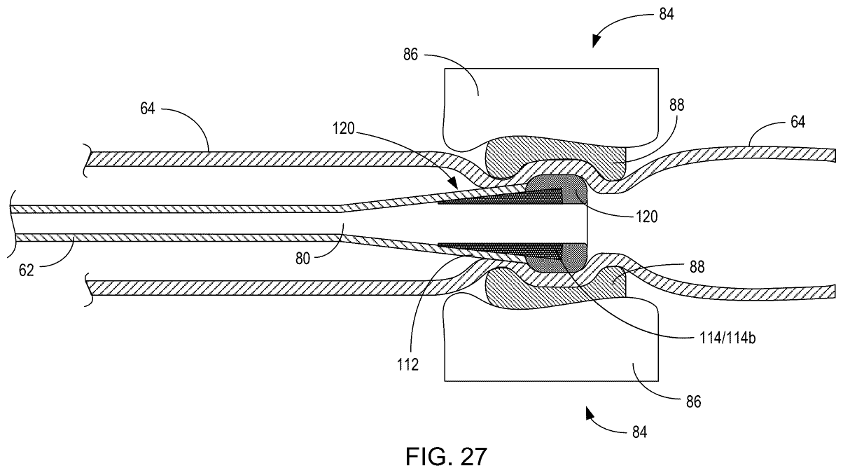

Referring now to FIG. 27, in embodiments where the instrument delivery device 82 includes a translation handle 84 for distally advancing the secondary catheter 62 through the extension tube 64, the stiffening component 114b may be specifically configured and shaped to engage with the translation handle 84 (through extension tube 64). For example, in the embodiment of FIG. 27, stiffening component 114b may be configured as a wedge-shaped stiffening member that extends out proximally past the proximal end 78 of secondary catheter 62, such as out past tapered portion 112, so that the stiffening component 114b provides engagement surfaces with which the gripping surfaces 88 of translation handle 84 can mate. The proximal end of stiffening component 114b may be shaped to directly mate with gripping surfaces 88 (through extension tube 64), or an additional cap 120 may be applied over the proximal end of stiffening component 114b to mate with gripping surfaces 88 (through extension tube 64)—with the cap 120 formed of a rigid or pliable material.

Beneficially, embodiments of the disclosure thus provide instrument delivery devices and associated vascular access systems that includes such delivery devices along with a catheter assembly that provides access to the vasculature of a patient. An instrument delivery device is configured to advance an instrument, such as a secondary catheter, into the catheter assembly and out past a distal tip of the primary indwelling catheter, to facilitate fluid delivery and/or blood withdrawal. A proximal end of the secondary catheter includes a retaining feature thereon that functions to retain the proximal end of the secondary catheter within the delivery device during advancement, with the retaining feature including a reinforcing structure that prevents deformation of the retaining feature, so as to ensure such retention of the secondary catheter.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. An instrument delivery device for introducing an instrument into a patient's vascular system via a catheter assembly including a primary catheter, the extension set comprising:
an extension tube comprising a proximal end, a distal end, and an outer tube wall that defines a tube lumen; and
an instrument disposed within the tube lumen and comprising a proximal end and a distal end, the instrument movable within the tube lumen to an advanced position where the distal end of the instrument extends into the primary catheter or beyond a distal tip thereof;
wherein the proximal end of the instrument comprises a retaining feature having a first outer diameter greater than a second outer diameter of the distal end of the instrument, the retaining feature comprising a reinforcing structure that prevents deformation of the retaining feature.

2. The instrument delivery device of claim 1, wherein the instrument comprises a secondary catheter defining a catheter lumen extending between the proximal end and the distal end thereof.

3. The instrument delivery device of claim 2, wherein the retaining feature comprises a tapered portion that flares radially outward from the second outer diameter to the first outer diameter, with the tapered portion having an increased wall thickness greater than a wall thickness of a remainder of the secondary catheter, thereby forming the reinforcing structure.

4. The instrument delivery device of claim 3, wherein the tapered portion having the increased wall thickness forms a funnel structure defining a shaped opening into the catheter lumen.

5. The instrument delivery device of claim 2, wherein the reinforcing structure comprises a stiffening component formed of a stiffening material having a stiffness greater than a stiffness of material from which the secondary catheter is formed.

6. The instrument delivery device of claim 5, wherein the stiffening component is affixed to the secondary catheter via one of adhesive bonding, welding, or swaging.

7. The instrument delivery device of claim 5, wherein the retaining feature comprises a tapered portion that flares radially outward from the second outer diameter to the first outer diameter, and wherein the stiffening component is positioned within the catheter lumen, in the tapered portion.

8. The instrument delivery device of claim 7, wherein the stiffening component defines an opening into the catheter lumen, the opening having a reduced opening area as compared to an opening area defined by the tapered portion.

9. The instrument delivery device of claim 7, wherein the stiffening component forms one of a wedge-shaped stiffening member, a ring-shaped stiffening member, and a frustoconical-shaped stiffening member.

10. The instrument delivery device of claim 5, wherein the stiffening component is positioned about the secondary catheter.

11. The instrument delivery device of claim 5, wherein at least one of the retaining feature and the stiffening component is tipped, to provide a radius or chamfer at an end thereof.

12. The instrument delivery device of claim 2, further comprising a distal connector positioned at the distal end of the extension tube to connect the extension tube to the catheter assembly.

13. The instrument delivery device of claim 2, further comprising a proximal connector positioned at the proximal end of the extension tube, the proximal connector configured to engage a fluid delivery device that delivers fluid to the catheter assembly through the secondary catheter.

14. The instrument delivery device of claim 13, wherein the instrument is configured to move between the retracted position and the advanced position responsive to injection of a flushing fluid through the proximal connector.

15. The instrument delivery device of claim 1, further comprising a translation handle coupled to an outer surface of the outer tube wall, wherein the translation handle is configured to move along the outer surface of the outer tube wall between a proximal position and a distal position to translate the distal end of the instrument between a retracted position and the advanced position.

16. The instrument delivery device of claim 15, wherein the translation handle engages the retaining feature through the outer tube wall, to provide for movement of the instrument between the retracted position and the advanced position.

17. A vascular access system comprising:
a catheter assembly including:
a catheter adapter comprising a distal adapter port, a proximal adapter port, and a side adapter port;
a primary catheter coupled to the distal adapter port and configured to be inserted into a patient's vasculature; and
a near patient connector coupled to the side adapter port via a first fluid conduit, the near patient connector comprising a distal connector port, a proximal connector port, and a side connector port; and
the instrument delivery device of claim 1, with the extension tube coupled to the near patient connector at the proximal connector port.

18. The vascular access system of claim 17, further comprising an extension set integrated with the near patient connector at the side connector port, the extension set comprising a second fluid conduit for providing fluid to the catheter assembly.

19. The vascular access system of claim 17, wherein the extension tube is integrated with the near patient connector at the proximal connector port.

20. The vascular access system of claim 17, wherein the extension tube is coupled to the near patient connector at the proximal connector port via one of a luer connector and a lock comprising a blunted cannular and a pair of locking arms.
